(19) United States
(12) Patent Application Publication
(10) Pub. No.: US 2006/0177404 A1
(43) Pub. Date: Aug. 10, 2006

(54) Cosmetic composition comprising at least one non-associative fixing polyurethane and at least one sulfonated polymer cosmetic hair treatment process

(76) Inventors: Francoise Pataut, Paris (FR); Charles Gringore, Asnieres-sur-seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/331,164

(22) Filed: Jan. 13, 2006

Related U.S. Application Data
(60) Provisional application No. 60/725,315, filed on Oct. 12, 2005.

(30) Foreign Application Priority Data
Jan. 13, 2005 (FR) 05 00367

(51) Int. Cl. A61K 8/81

(52) U.S. Cl. 424/70.15; 424/70.16; 424/70.17

(57) ABSTRACT

The present disclosure relates to a cosmetic hair treatment composition comprising at least one non-associative fixing polyurethane and at least one partially or totally neutralized or non-neutralized sulfonated polymer, comprising at least one unit derived from an ethylenically unsaturated monomer containing a non-aromatic sulfonic group.

This application claims benefit of U.S. Provisional Application No. 60/725,315, filed Oct. 12, 2005, the contents of which are incorporated herein by reference. This application also claims benefit of priority under 35 U.S.C. §119 to French Patent Application No. 05 00367, filed Jan. 13, 2005, the contents of which are also incorporated by reference.

The present disclosure relates to a cosmetic hair treatment composition comprising at least one non-associative fixing polyurethane and at least one particular type of sulfonated polymer, and also to a cosmetic treatment process using this composition.

Styling products, such as lacquers, mousses and gels, generally contain anionic or nonionic fixing polymers, such as polymers of polyurethane type. These products are usually used to structure the hairstyle and/or to give it lasting hold.

However, these products are not always easy to spread on the hair since the fixing of the hairstyle is often virtually immediate, and it is sometimes difficult to spread them onto the whole head of hair with the hands.

The present inventors have found, surprisingly, that by combining at least one non-associative fixing polyurethane and at least one associative or non-associative polymer comprising at least one unit derived from an ethylenically unsaturated monomer containing a non-aromatic sulfonic group, easy shaping of the hairstyle can be obtained, such as, for example, by simply working with the fingers, without obtaining the instantaneous hardening and setting conventionally encountered with the usual fixing products.

This composition may also have the benefit of spreading correctly on the hair, and may be applied either to dry hair or to wet hair.

In the case of application to wet hair, drying in the open air or with a hairdryer may be performed. This may result in a supple and natural hairstyle.

The present disclosure thus relates to a cosmetic hair treatment composition as described below.

The present disclosure also relates to a cosmetic process for treating the hair using the composition according to the present disclosure.

The present disclosure further relates to the use of the composition according to the present disclosure as a styling product.

Other characteristics, aspects and benefits of the present disclosure will emerge even more clearly on reading the description and the various examples that follow.

The present disclosure relates to a cosmetic hair treatment composition comprising, in a cosmetically acceptable medium, at least one non-associative fixing polyurethane and at least one partially or totally neutralized or non-neutralized sulfonated polymer, comprising at least one unit derived from an ethylenically unsaturated monomer containing a non-aromatic sulfonic group.

The term “ethylenically unsaturated monomer containing a non-aromatic sulfonic group,” as used herein, means a monomer containing at least one double bond and bearing in its structure a group SO₃X (with X denoting a hydrogen or ions of an alkali metal or alkaline-earth metal or ammonium ions or alternatively ions derived from an organic amine) not directly attached to an aromatic nucleus.

The term “cosmetically acceptable medium” as used herein means a medium that is compatible with the hair, but which also has a pleasant odor, appearance and feel.

The term “fixing polymer” as used herein means any polymer that can impart a shape to or that can hold a shape on a given head of hair.

For the purposes of the present disclosure, the term “non-associative fixing polyurethane” means fixing polycondensates comprising at least one polyurethane block capable of giving the hairstyle hold, which do not comprise in their structure any terminal or pendent fatty chains containing more than 10 carbon atoms. Examples of non-associative fixing polyurethanes are described in, for instance, European Patent Nos. EP 0 751 162, EP 0 637 600, EP 0 648 485, EP 0 656 021, and EP 0 619 111, French Patent No. FR 2 743 297, and International Patent Application Publication No. WO 94/03510.

The at least one non-associative fixing polyurethane used in accordance with the present disclosure may be soluble in the cosmetically acceptable medium, such as, for example, after neutralization with an organic or mineral base, or may form a dispersion in this medium. The dispersion may then comprise at least 0.05% of surfactant for dispersing and holding in dispersion the at least one non-associative fixing polyurethane.

According to the present disclosure, any type of surfactant may be used in the dispersion, such as, for example, a nonionic surfactant. In at least one embodiment, the mean size of the particles of the at least one non-associative fixing polyurethane in the dispersion may range from 0.1 to 1 micron.

In at least one embodiment, the at least one non-associative fixing polyurethanes used may be soluble in the medium.

By way of example, the at least one non-associative fixing polyurethane may be formed by an arrangement of blocks, this arrangement, for example, being obtained from:
(1) at least one compound which contains at least two active hydrogen atoms per molecule;
(2) at least one diol or a mixture of diols containing acid functions or salts thereof; and
(3) at least one di- or polyisocyanate.

In at least one embodiment, the compounds (1) may be chosen from diols, diamines, polyesterols and polyetherols, and mixtures thereof.

Among the compounds (1) that may be used according to at least one embodiment of the present disclosure, non-limiting mention may be made of linear polyethylene glycols and linear polypropylene glycols, such as, for example, those obtained by reacting ethylene oxide or propylene oxide with water or diethylene glycol or dipropylene glycol in the presence of sodium hydroxide as catalyst. These polyalkylene glycols generally have a molecular mass ranging from 600 to 20,000.

Other organic compounds that may be used according to the present disclosure, include, for example, those containing mercapto, amino, carboxyl or hydroxyl groups. Among examples of these organic compounds, non-limiting mention may be made of polyhydroxylated compounds such as polyetherdiols, polyesterdiols, polyacetaldiols, polyamidediols, polyesterpolyamidediols, poly(alkylene ether)diols, polythioetherdiols and polycarbonatediols.

In at least one embodiment, the polyetherdiols may include, for example, the products of condensation of ethylene oxide, propylene oxide or tetrahydrofuran, the grafted or block products of copolymerization or of condensation thereof, such as mixtures of condensates of ethylene oxide and of propylene oxide, and the products of polymerization of olefins, at high pressure, with alkylene oxide condensates. Suitable polyethers may be prepared, for example, by condensation of alkylene oxides and of polyhydric alcohols, such as ethylene glycol, 1,2-propylene glycol and 1,4-butanediol.

The polyesterdiols, polyesteramides and polyamidediols that may be used according to at least one embodiment of the present disclosure may be saturated and are obtained, for example, from the reaction of saturated or unsaturated polycarboxylic acids with polyhydric alcohols, diamines or polyamines. Adipic acid, succinic acid, phthalic acid, terephthalic acid and maleic acid may be used, for example, to prepare these compounds. Polyhydric alcohols that may be suitable for preparing polyesters include, for example, ethylene glycol, 1,2-propylene glycol, 1,4-butanediol, neopentyl glycol and hexanediol. Amino alcohols, for example ethanolamine, may also be used. Diamines that are suitable for preparing the polyesteramides may include ethylenediamine and hexamethylenediamine.

Suitable polyacetals may be prepared, for example, from 1,4-butanediol or hexanediol and formaldehyde. Suitable polythioethers may be prepared, for example, by condensation reaction between thioglycols alone or in combination with other glycols such as ethylene glycol, 1,2-propylene glycol or with other polyhydroxylated compounds. Polyhydroxylated compounds already containing urethane groups, natural polyols, which may be further modified, for example castor oil and carbohydrates, may also be used.

In at least one embodiment, the compound of group (1) may be a polyesterol, such as, for example, a polyesterdiol formed by the reaction of at least one (di)polyol (1ₐ) and at least one acid (1_{b}). The (di)polyol (1ₐ) may be chosen, for example, from the group comprising neopentyl glycol, 1,4-butanediol, hexanediol, ethylene glycol, diethylene glycol, propylene glycol, butylene glycol, neopentyl glycol and (di)polyethylene glycol. The acid (1_{b}) may be chosen, for example, from phthalic acid, isophthalic acid, adipic acid and (poly)lactic acid.

In at least one embodiment of the present disclosure, a hydroxycarboxylic acid such as dimethylolpropanoic acid (DMPA) or a 2,2-hydroxymethylcarboxylic acid may be used as compound (2). In general, compound (2) may be useful as a coupling block. Examples of compound (2) that may be used in embodiments of the present disclosure include those comprising at least one poly((α-hydroxycarboxylic diol) acid).

Among examples of compound (2) that may be used according to at least one embodiment of the present disclosure, non-limiting mention may be made of those chosen from 2,2-di(hydroxymethyl)acetic acid, 2,2-dihydroxymethylpropionic acid, 2,2-dihydroxymethylbutyric acid and 2,2-dihydroxymethylpentanoic acid.

Among examples of the di- or polyisocyanate (3) that may be used in at least one embodiment of the present disclosure, non-limiting mention be made of compounds chosen from hexamethylene diisocyanate, isophorone diisocyanate (IDPI), tolylene diisocyanate, 4,4′-diphenylmethane diisocyanate (DPMD) and 4,4′-dicyclohexylmethane diisocyanate (DCMD), methylenebis(p-phenyl diisocyanate), methylenebis(4-cyclohexyl isocyanate), toluene diisocyanates, 1,5-napthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanates, 2,2′-dichloro-4,4′-diisocyanatodiphenylmethane, 2,4-dibromo-1,5-diisocyanatonaphthalene, 1,4-butane diisocyanate, 1,6-hexane diisocyanate and 1,4-cyclohexane diisocyanate.

The at least one non-associative fixing polyurethane may be formed using an additional compound (4) that generally serves to lengthen its chain. These compounds (4) may be chosen from, for example, saturated or unsaturated glycols such as ethylene glycol, diethylene glycol, neopentyl glycol or triethylene glycol; amino alcohols such as ethanolamine, propanolamine or butanolamine; heterocyclic, aromatic, cycloaliphatic and aliphatic primary amines; diamines; carboxylic acids such as aliphatic, aromatic and heterocyclic carboxylic acids, for instance oxalic acid, succinic acid, glutaric acid, adipic acid, sebacic acid and terephthalic acid; and aminocarboxylic acids. In at least one embodiment of the present disclosure, the compounds (4) are aliphatic diols.

The at least one non-associative fixing polyurethane that may be used according to the present disclosure may also be formed from additional compounds (5) having a silicone skeleton, such as polysiloxanes, polyalkylsiloxanes or polyarylsiloxanes, including, for example, polyethylsiloxanes, polymethylsiloxanes and polyphenylsiloxanes, optionally comprising hydrocarbon-based chains grafted onto the silicon atoms.

In at least one embodiment of the present disclosure, the at least one non-associative fixing polyurethanes may comprise a base repeating unit corresponding to the general formula (I):
—O—B—O—CO—NH—R—NH—CO— (I)
wherein:
B is chosen from divalent C₁ to C₃₀ hydrocarbon-based groups, these groups being optionally substituted with a group comprising at least one functional group chosen from carboxylic acid functional groups and sulfonic acid functional groups, wherein the carboxylic acid and sulfonic acid functional groups are in free form or are partially or totally neutralized with a mineral or organic base, and
R is a divalent group chosen from aromatic, C₁ to C₂₀ aliphatic and C₁ to C₂₀ cycloaliphatic alkylene groups, wherein these groups are substituted or unsubstituted.

For example, the group R may be chosen from groups corresponding to the following formulae: 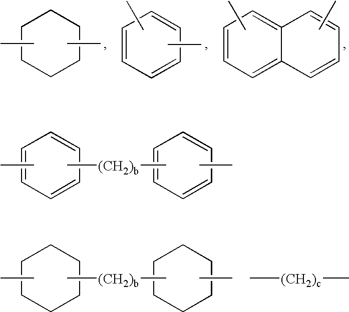
wherein b is an integer ranging from 0 to 3, and c is an integer ranging from 1 to 20, such as, for example, from 2 to 12.

In at least one embodiment, the group R is chosen from hexamethylene, 4,4′-biphenylenemethane, 2,4- and/or 2,6-tolylene, 1,5-naphthylene, p-phenylene and methylene-4,4-bis(cyclohexyl) groups and the divalent group derived from isophorone.

The at least one non-associative fixing polyurethane used in the present disclosure may also comprise at least one polysiloxane block, its base repeating unit corresponding, for example, to the general formula (II):
—O—P—O—CO—NH—R—NH—CO— (II)
wherein:
P is a polysiloxane segment, and
R is a divalent group chosen from aromatic, C₁ to C₂₀ aliphatic and C₁ to C₂₀ cycloaliphatic alkylene groups, these groups being substituted or unsubstituted.

In at least one embodiment of the present disclosure, the polysiloxane segment P corresponds to the general formula (III) below: 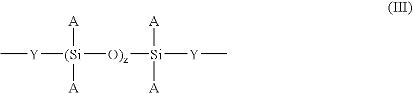
wherein:
the groups A, which may be identical or different, are chosen from monovalent C₁ to C₂₀ hydrocarbon-based groups that are free or substantially free of ethylenic unsaturation, and from aromatic groups,
Y is chosen from divalent hydrocarbon-based groups, and
z is an integer chosen such that the weight-average molecular mass of the polysiloxane segment ranges from 300 to 10,000.

In one embodiment, the divalent group Y may be chosen from the alkylene groups of formula —(CH₂)ₐ—, wherein a is chosen from an integer that may range from 1 to 10.

The groups A may be chosen from alkyl groups, such as, for example, methyl, ethyl, propyl, isopropyl, butyl, pentyl, hexyl, octyl, decyl, dodecyl and octadecyl groups; cycloalkyl groups, such as, for example, the cyclohexyl group; aryl groups, such as, for example, phenyl and naphthyl; arylalkyl groups, such as, for example, benzyl and phenylethyl, and also tolyl and xylyl groups.

Among examples of the at least one non-associative fixing polyurethanes that may be used according to the present disclosure, non-limiting mention may be made of the dimethylolpropionic acid/isophorone diisocyanate/neopentyl glycol/polyesterdiols copolymer (also known under the name polyurethane-1, INCI name) sold under the brand name Luviset® Pur by the company BASF, and the dimethylolpropionic acid/isophorone diisocyanate/neopentyl glycol/polyesterdiols/silicone diamine copolymer (also known under the name polyurethane-6, INCI name) sold under the brand name Luviset® Si Pur A by the company BASF.

The at least one non-associative fixing polyurethanes may be used in an amount ranging from 0.1% to 30% by weight, such as, for example, from 0.2% to 15% by weight or from 0.5% to 10% by weight, relative to the total weight of the hair treatment composition.

The sulfonated polymers in accordance with the present disclosure may be non-neutralized or partially or totally neutralized sulfonated associative or non-associative polymers, comprising at least one unit derived from an ethylenically unsaturated monomer containing a non-aromatic sulfonic group and optionally comprising at least one hydrophobic portion.

For the purposes of the present disclosure, the term “associative polymer” means any polymer containing at least one hydrophilic portion and at least one terminal or pendent fatty chain containing at least 10 carbon atoms. This type of polymer is capable of interacting with itself or with particular compounds such as surfactants, to lead to thickening of the medium.

For the purposes of the present disclosure, the term “partially neutralized” means sulfonic groups in salified form present in the polymer in a proportion of from 1% to 99%.

The hydrophobic portion optionally present in the polymers of the present disclosure may contain from 6 to 50 carbon atoms, such as, for example, from 6 to 22 carbon atoms, 6 to 18 carbon atoms, 10 to 18 carbon atoms or 12 to 18 carbon atoms.

In at least one embodiment, the at least one sulfonated polymer in accordance with the present disclosure is partially or totally neutralized with a mineral base (sodium hydroxide, potassium hydroxide or aqueous ammonia) or an organic base such as mono-, di- or triethanolamine, an aminomethylpropanediol, N-methylglucamine, basic amino acids, for instance arginine and lysine, and mixtures of these compounds.

The at least one sulfonated polymer in accordance with the present disclosure may have a number-average molecular weight ranging from 1000 to 20,000,000 g/mol, such as, for example, from 20,000 to 5,000,000 g/mol or from 100,000 to 1,500,000 g/mol.

The at least one sulfonated polymer according to the present disclosure may or may not be crosslinked.

In at least one embodiment of the present disclosure, the at least one sulfonated polymer is crosslinked.

When the sulfonated polymers are crosslinked, the crosslinking agents may be chosen from polyolefinically unsaturated compounds commonly used for the crosslinking of polymers obtained by free-radical polymerization.

Non-limiting mention may be made, for example, of divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylene-bismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allyl or vinyl ethers of polyfunctional alcohols, and also allylic esters of phosphoric and/or vinylphosphonic acid derivatives, or mixtures of these compounds.

Methylenebisacrylamide, allyl methacrylate or trimethylolpropane triacrylate (TMPTA) may be used in at least one embodiment of the present disclosure. The degree of crosslinking may range from 0.01 mol % to 10 mol %, such as, for example, from 0.2 mol % to 2 mol % relative to the polymer.

Among the ethylenically unsaturated monomers containing a non-aromatic sulfonic group that may be used according to the present disclosure, non-limiting mention may be made of vinylsulfonic acid, styrenesulfonic acid, (meth)acrylamido-(C₁-C₂₂)alkylsulfonic acids, and N—(C₁-C₂₂)alkyl(meth)acrylamido(C₁-C₂₂)alkylsulfonic acids, for instance undecylacrylamidomethanesulfonic acid, and also partially or totally neutralized forms thereof.

(Meth)acrylamido(C₁-C₂₂)alkylsulfonic acids such as, for example, acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, may also be used according to embodiments of the present disclosure.

2-Acrylamido-2-methylpropanesulfonic acid (AMPS), and also partially or totally neutralized forms thereof, may be used in at least one embodiment of the present disclosure.

The at least one sulfonated polymer in accordance with the present disclosure may be chosen from, for example, random amphiphilic AMPS polymers modified by reaction with a C₆-C₂₂ n-monoalkylamine or di-n-alkylamine, and such as those described in International Patent Application Publication No. WO 00/31154, the content of which is hereby incorporated by reference. These polymers may also contain other units derived from ethylenically unsaturated monomers not comprising a fatty chain.

These ethylenically unsaturated monomers not comprising a fatty chain may be chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The at least one sulfonated polymer may be chosen from amphiphilic copolymers of AMPS and of at least one ethylenically unsaturated hydrophobic monomer comprising at least one hydrophobic portion containing from 6 to 50 carbon atoms, such as, for example, 6 to 22 carbon atoms, 6 to 18 carbon atoms, 10 to 18 carbon atoms, or 12 to 18 carbon atoms.

These same copolymers may also contain at least one unit derived from ethylenically unsaturated monomers not comprising a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described in European Patent Application No. EP-A-750 899, U.S. Pat. No. 5,089,578, and in the following publications from Yotaro Morishima:
“Self-assembling amphiphilic polyelectrolytes and their nanostructures,” Chinese Journal of Polymer Science Vol. 18, No. 40, (2000), 323-336;
“Micelle formation of random copolymers of sodium 2-(acrylamido)-2-methylpropanesulfonate and a nonionic surfactant macromonomer in water as studied by fluorescence and dynamic light scattering,” Macromolecules 2000, Vol. 33, No. 10-3694-3704;
“Solution properties of micelle networks formed by nonionic moieties covalently bound to a polyelectrolyte: salt effects on rheological behavior,” Langmuir, 2000, Vol. 16, No. 12, 5324-5332;
“Stimuli responsive amphiphilic copolymers of sodium 2-(acrylamido)-2-methylpropanesulfonate and associative macromonomers,” Polym. Preprint, Div. Polym. Chem. 1999, 40(2), 220-221.

In at least one embodiment of the present disclosure, the ethylenically unsaturated hydrophobic monomers of these copolymers may be chosen from the acrylates or acrylamides of formula (I) below:

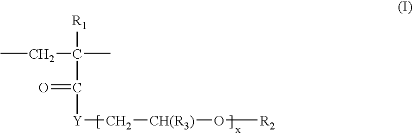

wherein:
R₁ and R₃, which may be identical or different, are chosen from hydrogen atoms and linear and branched C₁-C₆ alkyl radicals (for example, methyl);
Y is chosen from O and NH;
R₂ is chosen from hydrophobic hydrocarbon-based radicals containing at least from 6 to 50 carbon atoms, such as, for example, 6 to 22 carbon atoms, 6 to 18 carbon atoms, 10 to 18 carbon atoms, or 12 to 18 carbon atoms; and
x denotes the number of moles of alkylene oxide and ranges from 0 to 100.

The radical R₂ may be chosen from, for example, linear C₆-C₁₈ alkyl radicals (for example, n-hexyl, n-octyl, n-decyl, n-hexadecyl and n-dodecyl) and branched or cyclic C₆-C₁₈ alkyl radicals (for example, cyclododecane (C₁₂) or adamantane (C₁₀)); C₆-C₁₈ alkylperfluoro radicals (for example, the group of formula —(CH₂)₂—(CF₂)₉—CF₃); the cholesteryl radical (C₂₇) or a cholesterol ester residue, for instance the cholesteryl oxyhexanoate group; aromatic polycyclic groups, for instance naphthalene or pyrene. In at least one embodiment of the present disclosure, the radical R₂ is chosen from linear alkyl radicals, such as the n-dodecyl radical.

According to at least one embodiment of the present disclosure, the monomer of formula (I) comprises at least one alkylene oxide unit (x≧1) and a polyoxyalkylenated chain. The polyoxyalkylenated chain may comprise, for example, ethylene oxide units and/or propylene oxide units. In at least one embodiment, the polyoxyalkylenated chain comprises ethylene oxide units. The number of oxyalkylene units may range from 3 to 100, such as, for example, from 3 to 50 or from 7 to 25.

Among these polymers, non-limiting mention may be made of:
crosslinked or noncrosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer, such as those described in European Patent Application No. EP-A-750 899;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Non-limiting mention may also be made of copolymers of totally neutralized AMPS and of dodecyl methacrylate, and also crosslinked and noncrosslinked copolymers of AMPS and of n-dodecylmethacrylamide, such as those described in the Morishima articles mentioned above.

Further mention may be made of the copolymers consisting of 2-acrylamido-2-methylpropanesulfonic acid (AMPS) units of formula (II) below:

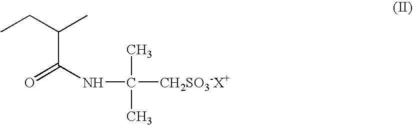

wherein X⁺ is chosen from protons, alkali metal cations, alkaline-earth metal cations and ammonium ions,

and of units of formula (III) below:

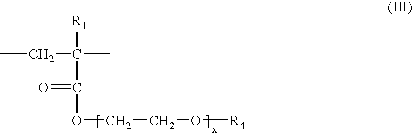

wherein:
x is an integer ranging from 3 to 100, such as, for example, from 5 to 80 or from 7 to 25;
R₁ has the same meaning as that given above in formula (I); and
R₄ is chosen from linear and branched C₆-C₂₂ alkyl groups, such as, for example, C₁₀-C₂₂ alkyl groups.

In at least one embodiment of the present disclosure, the sulfonated polymers are those for which x is 25, R₁ is methyl and R₄ is n-dodecyl. These polymers are described in the Morishima articles mentioned above.

In at least one embodiment, the polymers for which X⁺ is chosen from sodium and ammonium are used.

The at least one sulfonated polymers in accordance with the present disclosure may be obtained according to the standard free-radical polymerization processes in the presence of at least one initiators such as, for example, azobisisobutyronitrile (AIBN), azobisdimethylvaleronitrile, ABAH (2,2-azobis[2-amidinopropane]hydrochloride), organic peroxides such as dilauryl peroxide, benzoyl peroxide, tert-butyl hydroperoxide, etc., mineral peroxide compounds such as potassium persulfate or ammonium persulfate, or H₂O₂ optionally in the presence of reducing agents.

The amphiphilic polymers may be obtained, for example, by free-radical polymerization in tert-butanol medium in which they precipitate.

Using precipitation polymerization in tert-butanol, it may be possible to obtain a beneficial size distribution of the polymer particles.

The size distribution of the polymer particles may be determined, for example, by laser diffraction or image analysis.

In at least one embodiment, the size distribution for this type of polymer, determined by image analysis, may be as follows: 60.2% less than 423 microns, 52.0% less than 212 microns, 26.6% less than 106 microns, 2.6% less than 45 microns and 26.6% greater than 850 microns.

The reaction may be performed at a temperature ranging from 0 to 150° C., such as from 10 to 100° C., either at atmospheric pressure or under reduced pressure. It may also be performed under an inert atmosphere, such as, for example, under nitrogen.

According to this process, 2-acrylamido-2-methylpropanesulfonic acid (AMPS) or a sodium or ammonium salt thereof may be polymerized with a (meth)acrylic acid ester and
a C₁₀-C₁₈ alcohol oxyethylenated with 8 mol of ethylene oxide (Genapol® C-080 from the company Hoechst/Clariant),
a C₁₁ oxo alcohol oxyethylenated with 8 mol of ethylene oxide (Genapol® UD-080 from the company Hoechst/Clariant),
a C₁₁ oxo alcohol oxyethylenated with 7 mol of ethylene oxide (Genapol® UD-070 from the company Hoechst/Clariant),
a C₁₂-C₁₄ alcohol oxyethylenated with 7 mol of ethylene oxide (Genapol® LA-070 from the company Hoechst/Clariant),
a C₁₂-C₁₄ alcohol oxyethylenated with 9 mol of ethylene oxide (Genapol® LA-090 from the company Hoechst/Clariant),
a C₁₂-C₁₄ alcohol oxyethylenated with 11 mol of ethylene oxide (Genapol® LA-110 from the company Hoechst/Clariant),
a C₁₆-C₁₈ alcohol oxyethylenated with 8 mol of ethylene oxide (Genapol® T-080 from the company Hoechst/Clariant),
a C₁₆-C₁₈ alcohol oxyethylenated with 15 mol of ethylene oxide (Genapol® T-150 from the company Hoechst/Clariant),
a C₁₆-C₁₈ alcohol oxyethylenated with 11 mol of ethylene oxide (Genapol® T-110 from the company Hoechst/Clariant),
a C₁₆-C₁₈ alcohol oxyethylenated with 20 mol of ethylene oxide (Genapol® T-200 from the company Hoechst/Clariant),
a C₁₆-C₁₈ alcohol oxyethylenated with 25 mol of ethylene oxide (Genapol® T-250 from the company Hoechst/Clariant), or
a C₁₈-C₂₂ alcohol oxyethylenated with 25 mol of ethylene oxide and/or a C₁₆-C₁₈ iso alcohol oxyethylenated with 25 mol of ethylene oxide.

The percentage molar concentration of the units of formula (II) and of the units of formula (III) in the polymers according to the present disclosure will vary as a function of the desired cosmetic use and of the desired Theological properties of the formulation. For example, it may range from 0.1 mol % to 99.9 mol %.

For example, for the most hydrophobic polymers, the molar proportion of units of formula (I) or (III) may range from 50.1% to 99.9%, such as from 70% to 95% or from 80% to 90%.

For the sparingly hydrophobic polymers, the molar proportion of units of formula (I) or (III) may range from 0.1% to 50%, such as from 5% to 25% or from 10% to 20%.

The monomer distribution in the polymers of the present disclosure may be, for example, alternating, block (including multiblock) or random.

According to at least one embodiment of the present disclosure, the polymers may contain heat-sensitive pendant chains and the aqueous solution thereof may have a viscosity that, beyond a certain threshold temperature, increases or remains virtually constant as the temperature increases.

For example, the polymers may include those whose aqueous solution has a viscosity that is low below a first threshold temperature and that, above this first threshold temperature, increases to a maximum as the temperature increases, and that, above a second threshold temperature, decreases again as the temperature increases. In at least one embodiment of the present disclosure, the viscosity of the polymer solutions below the first threshold temperature ranges from 5% to 50%, such as, for example, from 10% to 30% of the maximum viscosity at the second threshold temperature.

In water, these polymers may lead to a phenomenon of demixing by heating, reflected by curves showing, as a function of the temperature and the concentration, a minimum known as the LCST (Lower Critical Solution Temperature).

The viscosities (measured at 25° C. using a Brookfield viscometer with a No 7 needle) of the aqueous 1% solutions may range from 20,000 mpa.s to 100,000 mPa.s, such as, for example, from 60,000 mPa.s to 70,000 mPa.s.

The at least one sulfonated polymer may be present in an amount ranging from 0.01% to 20% by weight, such as, for example, from 0.1% to 10% by weight or from 0.2% to 5% by weight relative to the total weight of the hair treatment composition.

In at least one example of the present disclosure, the at least one non-associative fixing polyurethane/at least one sulfonated polymer weight ratio is greater than 1.30.

The cosmetically acceptable medium may comprise water and/or a cosmetically acceptable solvent such as a C₁-C₄ lower alcohol, such as ethanol, isopropanol, tert-butanol or n-butanol; alkylene polyols, for instance propylene glycol; polyol ethers; and mixtures thereof.

The composition according to the present disclosure may also comprise at least one common cosmetic additive chosen from adhesives, reducing agents, for instance thiols, fatty substances, thickeners other than the at least one sulfonated polymer of the present disclosure, nacreous agents, softeners, antifoams, sunscreens, antiperspirants, acidifying agents, basifying agents, dyes, pigments, fragrances, preserving agents, surfactants, volatile or non-volatile silicones, plant, mineral or synthetic oils, proteins and vitamins, and mixtures thereof.

A person skilled in the art will take care to select the optional additives and the amount thereof such that they do not harm the properties of the compositions of the present disclosure.

The optional additives may be present in the composition according to the present disclosure in an amount ranging from 0 to 10% by weight relative to the total weight of the composition.

The compositions in accordance with the present disclosure may be in the form of a gel, a liquid or a mousse and may be used in rinse-out or leave-in application. They may be packaged in a tube or a vaporizer or in an aerosol device that is common in cosmetics.

The propellants used in the aerosol systems according to the present disclosure may be liquefied or compressed gases and are chosen in particular from C₃-C₅ alkanes, DME and fluoroalkanes such as Dymel 152A, or mixtures thereof.

The compositions in accordance with the present disclosure may be used as hair fixing and/or holding compositions, haircare compositions, shampoos, hair conditioning compositions, such as compositions for giving the hair softness, or alternatively hair makeup compositions.

The present disclosure also relates to a cosmetic process for treating the hair that comprises applying an effective amount of a composition as described above to the hair, and in optionally rinsing it out after an optional leave-in time.

According to one embodiment of the present disclosure, the composition may be used as a leave-in styling product.

Other than in the operating examples, or where otherwise indicated, all numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where the term “between” is used, the ranges defined include the stated end points.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The example that follows serves to illustrate the invention without, however, being limiting in nature.

### EXAMPLE

A styling product in the form of an aerosol spray was prepared. The amounts are indicated as weight percentages:
Luviset® Si PUR sold by the company BASF 2%
Crosslinked polyacrylamidomethylpropanesulfonic acid partially neutralized with aqueous ammonia, sold under the name ARISTOFLEX HMS by the company Clariant 1%
Ethanol 23%
Dimethyl ether 35%
Water qs 100%

This styling product was sprayed onto the hair and the hairstyle was shaped. Good hairstyle hold was obtained.

What is claimed is:

1. A cosmetic hair treatment composition, comprising, in a cosmetically acceptable medium:
at least one non-associative fixing polyurethane; and
at least one partially or totally neutralized or non-neutralized sulfonated polymer comprising at least one unit derived from an ethylenically unsaturated monomer containing a non-aromatic sulfonic group.

2. A composition according to claim 1, wherein the at least one non-associative fixing polyurethane comprises a base repeating unit of formula (I):
—O—B—O—CO—NH—R—NH—CO— (I)
wherein:
B is chosen from divalent C₁ to C₃₀ hydrocarbon-based groups which are optionally substituted with a group comprising at least one functional group chosen from carboxylic acid functional groups and sulfonic acid functional groups, wherein the carboxylic acid and sulfonic acid functional groups are in free form or are partially or totally neutralized with a mineral or organic base, and
R is a divalent group chosen from aromatic, C₁ to C₂₀ aliphatic and C₁ to C₂₀ cycloaliphatic alkylene groups, wherein the aromatic, C₁ to C₂₀ aliphatic and C₁ to C₂₀ cycloaliphatic alkylene groups are optionally substituted.

3. A composition according to claim 1, wherein the at least one non-associative fixing polyurethane comprises a base repeating unit of formula (II):
—O—P—O—CO—NH—R—NH—CO— (II)
wherein:
P is a polysiloxane segment, and
R is a divalent group chosen from aromatic, C₁ to C₂₀ aliphatic or C₁ to C₂₀ cycloaliphatic alkylene groups, wherein the aromatic, C₁ to C₂₀ aliphatic or C₁ to C₂₀ cycloaliphatic alkylene groups are optionally substituted.

4. A composition according to claim 1, wherein the at least one non-associative fixing polyurethane is a dimethylolpropionic acid/isophorone diisocyanate/neopentyl glycol/polyester diol copolymer.

5. A composition according to claim 1, wherein the at least one non-associative fixing polyurethane is a dimethylolpropionic acid/isophorone diisocyanate/neopentyl glycol/polyester diol/silicone-based diamine copolymer.

6. A composition according to claim 1, wherein the ethylenically unsaturated monomer containing a non-aromatic sulfonic group is chosen from vinylsulfonic acid, (meth)acrylamido(C₁-C₂₂)alkylsulfonic acids and N—(C₁-C₂₂)alkyl(meth)acrylamido(C₁-C₂₂)alkylsulfonic acids, and the partially and totally neutralized forms thereof.

7. A composition according to claim 6, wherein the ethylenically unsaturated monomer containing a non-aromatic sulfonic group is chosen from acrylamidomethanesulfonic acid, acrylamidoethanesulfonic acid, acrylamidopropane-sulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid and 2-acrylamido-2,6-dimethyl-3-heptanesulfonic acid, and the partially and totally neutralized forms thereof.

8. A composition according to claim 7, wherein the ethylenically unsaturated monomer containing a non-aromatic sulfonic group is chosen from 2-acrylamido-2-methylpropanesulfonic acid (AMPS), and the partially and totally neutralized forms thereof.

9. A composition according to claim 8, wherein the at least one sulfonated polymer further comprises at least one unit derived from an ethylenically unsaturated monomer not comprising a fatty chain.

10. A composition according to claim 9, wherein the ethylenically unsaturated monomer not comprising a fatty chain is chosen from:
(meth)acrylic acids and β-substituted alkyl derivatives thereof, and esters thereof obtained with monoalcohols or monoalkylene or polyalkylene glycols;
(meth)acrylamides, vinylpyrrolidone, maleic anhydride, itaconic acid and maleic acid; and
mixtures thereof.

11. A composition according to claim 1, wherein the at least one sulfonated polymer is chosen from amphiphilic copolymers of AMPS and at least one ethylenically unsaturated hydrophobic monomer comprising at least one hydrophobic portion containing from 6 to 50 carbon atoms.

12. A composition according to claim 11, wherein the at least one hydrophobic portion contains from 6 to 22 carbon atoms.

13. A composition according to claim 12, wherein the at least one hydrophobic portion contains from 6 to 18 carbon atoms.

14. A composition according to claim 13, wherein the at least one hydrophobic portion contains from 10 to 18 carbon atoms.

15. A composition according to claim 14, wherein the at least one hydrophobic portion contains from 12 to 18 carbon atoms.

16. A composition according to claim 11, wherein the at least one ethylenically unsaturated hydrophobic monomer is chosen from acrylates and acrylamides of formula (I) below: 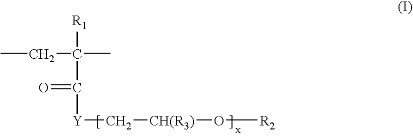
wherein:
R₁ and R₃, which may be identical or different, are chosen from hydrogen atoms and linear and branched C₁-C₆ alkyl radicals;
Y is chosen from O and NH;
R₂ is chosen from hydrophobic hydrocarbon-based radicals containing at least 6 to 50 carbon atoms;
x ranges from 0 to 100.

17. A composition according to claim 16, wherein at least one of R₁ and R₃ is chosen from methyl.

18. A composition according to claim 16, wherein R₂ is chosen from hydrophobic hydrocarbon-based radicals containing 12 to 18 carbon atoms.

19. A composition according to claim 16, wherein R₂ is chosen from linear, branched and cyclic C₆-C₁₈ alkyl radicals; C₆-C₁₈ perfluoroalkyl radicals; the cholesteryl radical; a cholesterol ester; and polycyclic aromatic groups.

20. A composition according to claim 16, wherein the monomer of formula (I) further comprises at least one alkylene oxide unit (x≧1).

21. A composition according to claim 16, wherein the monomer of formula (I) further comprises at least one polyoxyalkylenated chain.

22. A composition according to claim 21, wherein the at least one polyoxyalkylenated chain comprises ethylene oxide units and/or propylene oxide units.

23. A composition according to claim 22, wherein the at least one polyoxyalkylenated chain consists solely of ethylene oxide units.

24. A composition according to claim 16, wherein the number of oxyalkylene units ranges from 3 to 100.

25. A composition according to claim 24, wherein the number of oxyalkylene units ranges from 3 to 50.

26. A composition according to claim 25, wherein the number of oxyalkylene units ranges from 7 to 25.

27. A composition according to claim 11, wherein the at least one sulfonated polymer is chosen from:
crosslinked and non-crosslinked, neutralized and non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer; and
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units relative to the polymer.

28. A composition according to claim 11, wherein the at least one sulfonated polymer is chosen from:
non-crosslinked copolymers of partially or totally neutralized AMPS and n-dodecyl methacrylate; and
crosslinked or non-crosslinked copolymers of partially or totally neutralized AMPS and of n-dodecylmethacrylamide.

29. A composition according to claim 11, wherein the at least one sulfonated polymer is chosen from copolymers of 2-acrylamido-2-methylpropanesulfonic acid (AMPS) units of formula (II) below: 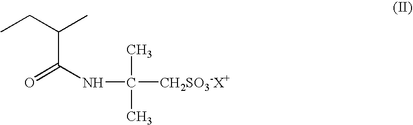
wherein X⁺ is chosen from protons, alkali metal cations, alkaline-earth metal cations and ammonium ions,
and of units of formula (III) below: 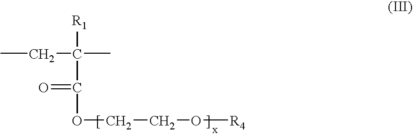
wherein:
x is an integer ranging from 3 to 100;
R₁ is chosen from hydrogen atoms and linear and branched C₁-C₆ alkyl radicals; and
R₄ is chosen from linear and branched C₆-C₂₂ alkyl radicals.

30. A composition according to claim 29, wherein x is an integer ranging from 7 to 25.

31. A composition according to claim 29, wherein R₄ is chosen from linear and branched C₁₀-C₂₂ alkyl radicals.

32. A composition according to claim 29, wherein x is 25, R₁ is methyl and R₄ is n-dodecyl.

33. A composition according to claim 16, wherein the percentage molar proportion of the units of formula (I) or of the units of formula (III) in the polymers ranges from 50.1% to 99.9%.

34. A composition according to claim 16, wherein the percentage molar proportion of the units of formula (I) or of the units of formula (III) in the polymers ranges from 0.1% to 50%.

35. A composition according to claim 1, wherein the at least one sulfonated polymer is partially or totally neutralized with a mineral or organic base.

36. A composition according to claim 1, wherein the at least one sulfonated polymer has a number-average molecular weight ranging from 1000 to 20,000,000 g/mol.

37. A composition according to claim 36, wherein the number-average molecular weight ranges from 20,000 to 5,000,000 g/mol.

38. A composition according to claim 37, wherein the number-average molecular weight ranges from 100,000 to 1,500,000 g/mol.

39. A composition according to claim 1, wherein an aqueous solution containing 1% by weight of the at least one sulfonated polymer has, at a temperature of 25° C., a viscosity, measured using a Brookfield viscometer with a No. 7 needle, ranging from 20,000 mPa.s to 100,000 mPa.s.

40. A composition according to claim 1, wherein the at least one sulfonated polymer is prepared by free-radical polymerization by precipitation from tert-butanol.

41. A composition according to claim 1, wherein the at least one sulfonated polymer is crosslinked or non-crosslinked.

42. A composition according to claim 41, wherein the at least one sulfonated polymer is crosslinked.

43. A composition according to claim 42, further comprising at least one crosslinking agent is chosen from polyolefinically unsaturated compounds.

44. A composition according to claim 43, wherein the at least one crosslinking agent is chosen from methylenebisacrylamide, allyl methacrylate and trimethylolpropane triacrylate (TMPTA).

45. A composition according to claim 42, wherein the degree of crosslinking ranges from 0.01 mol % to 10 mol % relative to the polymer.

46. A composition according to claim 45, wherein the degree of crosslinking ranges from 0.2 mol % to 2 mol % relative to the polymer.

47. A composition according to claim 1, wherein the at least one non-associative fixing polyurethane is present in an amount ranging from 0.1% to 30% by weight relative to the total weight of the composition.

48. A composition according to claim 47, wherein the at least one non-associative fixing polyurethane is present in an amount ranging from 0.5% to 10% by weight relative to the total weight of the composition.

49. A composition according to claim 1, wherein the at least one sulfonated polymer is present in an amount ranging from 0.01 % to 20% by weight relative to the total weight of the composition.

50. A composition according to claim 49, wherein the at least one sulfonated polymer is present in an amount ranging from 0.2% to 5% by weight relative to the total weight of the composition.

51. A composition according to claim 1, wherein the cosmetically acceptable medium comprises water and/or a cosmetically acceptable solvent.

52. A composition according to claim 51, wherein the cosmetically acceptable solvent is chosen from C₁-C₄ lower alcohols, alkylene polyols and polyol ethers, and mixtures thereof.

53. A composition according to claim 51, wherein the cosmetically acceptable solvent is chosen from ethanol, isopropanol, tert-butanol and propylene glycol.

54. A composition according to claim 1, further comprising at least one cosmetic additive chosen from adhesives, reducing agents, fatty substances, thickeners, softeners, nacreous agents, antifoams, sunscreens, antiperspirants, acidifying agents, basifying agents, dyes, pigments, fragrances, preserving agents, surfactants, volatile and non-volatile silicones, plant and mineral oils, proteins and vitamins, and mixtures thereof.

55. A composition according to claim 1, wherein the composition is in the form of a gel, a liquid or a mousse.

56. A composition according to claim 1, wherein the composition is packaged in a tube, a vaporizer or an aerosol device.

57. A cosmetic hair treatment process, comprising applying to the hair a cosmetic hair treatment composition, and optionally rinsing out the composition after an optional leave-in time, wherein the composition comprises, in a cosmetically acceptable medium:
at least one non-associative fixing polyurethane; and
at least one partially or totally neutralized or non-neutralized sulfonated polymer comprising at least one unit derived from an ethylenically unsaturated monomer containing a non-aromatic sulfonic group.

58. A method of styling hair, comprising applying to the hair a cosmetic hair treatment composition and then styling the hair, wherein the composition comprises, in a cosmetically acceptable medium:
at least one non-associative fixing polyurethane; and
at least one partially or totally neutralized or non-neutralized sulfonated polymer comprising at least one unit derived from an ethylenically unsaturated monomer containing a non-aromatic sulfonic group.
